SEQUENCE LISTING (118 sequences)

SEQ ID NO: 1 - protein, 14 aa, Chlamydia pneumoniae
KPKESKTDSVERWS

SEQ ID NO: 2 - protein, 11 aa, Chlamydia pneumoniae
SSNSSSSTSRS

SEQ ID NO: 3 - protein, 8 aa, Chlamydia pneumoniae
GSKQQGSS

SEQ ID NO: 4 - protein, 8 aa, Chlamydia pneumoniae
GKAGQQQG

SEQ ID NO: 5 - protein, 9 aa, Chlamydia pneumoniae
PSETSTTEK

SEQ ID NO: 6 - protein, 8 aa, Chlamydia pneumoniae
KPADGSDV

SEQ ID NO: 7 - protein, 11 aa, Chlamydia pneumoniae
NGQKKPLYLYG

SEQ ID NO: 8 - protein, 19 aa, Chlamydia pneumoniae
SDVPNPGTTVGGSKQQGSS

SEQ ID NO: 9 - protein, 15 aa, Chlamydia pneumoniae
HMFNTENPDSQAAQQ

SEQ ID NO: 10 - protein, 9 aa, Chlamydia pneumoniae
DDAENETAS

SEQ ID NO: 11 - protein, 16 aa, Chlamydia pneumoniae
KEEDKKRREASDAKNE

SEQ ID NO: 12 - protein, 16 aa, Homo sapiens
AEEDRRKKERVEAVNM

SEQ ID NO: 13 - protein, 22 aa, Chlamydia pneumoniae
KKHSFSTKPPSNNGSSEDHIEE

SEQ ID NO: 14 - protein, 14 aa, Chlamydia pneumoniae
YTVTSGSKGDAVFE

SEQ ID NO: 15 - protein, 11 aa, Chlamydia pneumoniae
TSSEGTRTTPS

SEQ ID NO: 16 - protein, 8 aa, Chlamydia pneumoniae
SEHKKSSK

SEQ ID NO: 17 - protein, 14 aa, Chlamydia pneumoniae
KDVASGKEQKIRIE

SEQ ID NO: 18 - protein, 16 aa, Chlamydia pneumoniae
ERNTTIPTQKKQIFST

SEQ ID NO: 19 - protein, 16 aa, Chlamydia pneumoniae
YFNDSQRASSTKDAGR

SEQ ID NO: 20 - protein, 15 aa, Chlamydia pneumoniae
EEFKKQEGIDLSKDN

SEQ ID NO: 21 - protein, 12 aa, Chlamydia pneumoniae
NAKGGPNINTED

SEQ ID NO: 22 - protein, 16 aa, Chlamydia pneumoniae
GERPMAKDNKEIGRFD

SEQ ID NO: 23 - protein, 14 aa, Chlamydia pneumoniae
TEIEMKEKKDRVDD

SEQ ID NO: 24 - protein, 14 aa, Homo sapiens
SDVEVNEKKDRVTD

SEQ ID NO: 25 - protein, 11 aa, Chlamydia pneumoniae
EDSTSDYDKEK

SEQ ID NO: 26 - protein, 11 aa, Homo sapiens
DVTTSEYEKEK

SEQ ID NO: 27 - protein, 7 aa, Chlamydia pneumoniae
DDKSSSA

SEQ ID NO: 28 - protein, 15 aa, Chlamydia pneumoniae
KKQIEDSTSDYVSEE

SEQ ID NO: 29 - protein, 12 aa, Chlamydia pneumoniae
SSYFSTNPETQE

SEQ ID NO: 30 - protein, 16 aa, Chlamydia pneumoniae
EKVGKNGSITVEEADK

SEQ ID NO: 31 - protein, 15 aa, Chlamydia pneumoniae
SKTADKAGDGTTTAT

SEQ ID NO: 32 - protein, 10 aa, Chlamydia pneumoniae
KREEEEATAR

SEQ ID NO: 33 - protein, 10 aa, Mouse
ERSAAETVTK

SEQ ID NO: 34 - protein, 11 aa, Chlamydia pneumoniae
DTAKKKQDRAE

SEQ ID NO: 35 - protein, 11 aa, Mouse
EKSQGKVLQAT

SEQ ID NO: 36 - protein, 10 aa, Chlamydia pneumoniae
GTGKRTDDGT

SEQ ID NO: 37 - protein, 10 aa, Mouse
GSGGKGKSGE

SEQ ID NO: 38 - protein, 25 aa, Chlamydia pneumoniae
RRNKQPVEQKSRGAFCDKEFYPCEE

SEQ ID NO: 39 - protein, 19 aa, Chlamydia pneumoniae
DMRPGDKKVFTVEFCPQRR

SEQ ID NO: 40 - protein, 19 aa, Chlamydia pneumoniae
SSDPETTPTSDGKVWKIDR

SEQ ID NO: 41 - protein, 20 aa, Chlamydia pneumoniae
TSESNCGTCTSCAETTTHWK

SEQ ID NO: 42 - protein, 11 aa, Chlamydia pneumoniae
KLGSKESVEFS

SEQ ID NO: 43 - protein, 16 aa, Chlamydia pneumoniae
TVYRICVTNRGSAEDT

SEQ ID NO: 44 - protein, 11 aa, Chlamydia pneumoniae
EYSISVSNPGD

SEQ ID NO: 45 - protein, 100 aa, Chlamydia trachomatis
MKKLLKSVLVFAALSSASSLQALPVGNPAEPSLMIDGILWEGFGGDPCDPCTTWCDAISMRMGYYGDFVFDRVLQTDVNKEFQMGAKPTTATGNAAAPST

SEQ ID NO: 46 - protein, 100 aa, Chlamydia trachomatis
MKKLLKSVLVFAALSSASSLQALPVGNPAEPSLMIDGILWEGFGGDPCDPCTTWCDAISMIMGYYGDFVFDRVLKTDVNKEFQMGAKPTTTTGNAAAPST

SEQ ID NO: 47 - protein, 100 aa, Chlamydia trachomatis
MKKLLKSVLVFAALSSASSLQALPVGNPAEPSLMIDGILWEGFGGDPCDPCTTWCDAISMRMGYYGDFVFDRVLETDVNKEFHMGAKPTTDTGNSAAPLT

SEQ ID NO: 48 - protein, 100 aa, Chlamydia trachomatis
MKKLLKSVLVFAALSSASSLQALPVGNPAEPSLMIDGILWEGFGGDPCDPCTTWCDAISMIMGYYGDFVFDRVLKTDVNKEFHMGDKPTSTTGNATAPTT

SEQ ID NO: 49 - protein, 100 aa, Chlamydia trachomatis
MKKLLKSVLVFAALSSASSLQALPVGNPAEPSLMIDGILWEGFGGDPCDPCTTWCDAISMIMGYYGDFVFDRVLKTDVNKEFHMGDKPTATTGNAAAPST

SEQ ID NO: 50 - protein, 101 aa, Chlamydia trachomatis
MKKLLKSVLVFAALSSASSLQALPVGNPAEPSLMIDGILWEGFGGDPCDPCTTWCDAISMIMGYYGDFVFDRVLKTDVNKEFKMGEALAGSTGNTTSTLSK

SEQ ID NO: 51 - protein, 103 aa, Chlamydia trachomatis
MKKLLKSVLVFAALSSASSLQALPVGNPAEPSLMIDGILWEGFGGDPCDPCTTWCDAISMVMGYYGDFVFDRVLKTDVNKEFQMGAAPTTSDVAAGLQNDPTI

SEQ ID NO: 52 - protein, 102 aa, Chlamydia trachomatis
MKKLLKSVLVFAALSSASSLQALPVGNPAEPSLMIDGILWEGFGGDPCDPCTTWCDAISMRMGYYGDFVFDRVLKTDVNKEFQMGAAPTTRDVAGLEKDPVV

SEQ ID NO: 53 - protein, 97 aa, Chlamydia trachomatis
MKKLLKSVLVFAALSSASSLQALPVGNPAEPSLMIDGILWEGFGGDPCAPCTTWCDAISMVMGYYGDFVFDRVLKTDVNKEFQMGAAPTTNDAAPKT

SEQ ID NO: 54 - protein, 102 aa, Chlamydia trachomatis
MKKLLKSVLVFAALSSASSLQALPVGNPAEPSLMIDGILWEGFGGDPCDPCTTWCDAISMVMGYYGDFVFDRVLKTDVNKEFQMGAEPTTSDTAGLSNDPTT

SEQ ID NO: 55 - protein, 100 aa, Chlamydia trachomatis
MKKLLKSVAVFVAGSSASSLHALPVGNPAEPSLMIDGILWEGFGGDPCDPCTTWCDAISMRMGLYLDFVFDRVLKTDVNKQFEMGAAPTGDADLTTAPTP

SEQ ID NO: 56 - protein, 99 aa, Chlamydia pneumoniae
MKKLLKAVLAFAFAGSVGSLQALPVGNPASDSLLIDGTIWEGAAGDPCDPATTWCDAISLRAGFYGDFVYDIVLKVDAPKTFSMGAKPTTTGNGSAAAN

SEQ ID NO: 57 - protein, 100 aa, Chlamydia trachomatis
CTARENPAYGRHMQDAEMFTNAAYMALINWDRFDVFCTLGATSGYLKGNSASFNLVGLFGDNENHATVSDSKLVPNMSLDQSVVELYTDTTFAWSAGARA

SEQ ID NO: 58 - protein, 65 aa, Chlamydia trachomatis
LTARENPAYGRHMQDAEMFTNCAYMALINWDRFDVFCTLGASSGYLKGNSASFNLVGLFGNNENQ

SEQ ID NO: 59 - protein, 134 aa, Chlamydia trachomatis
CTARENPAYGRHMQDAEMFTNCAYMALINWDRFDVFCTLGATSGYLKGNSASFNLVGLFGDNENQKTVKAESVPNMSFDQSVVELYTDTTFAWSVGARATKVSNGTFVPNMSLDQSVVELYTDTAFAWSVGARA

SEQ ID NO: 60 - protein, 99 aa, Chlamydia trachomatis
LTARENPAYGRHMQDAEMFTNCAYMALINWDRFDVFCTLGASSGYLKGNSASFNLVGLFGDNENQSTVKTNSVPNMSLDQSVVELYTDTAFSWSVGARA

SEQ ID NO: 61 - protein, 99 aa, Chlamydia trachomatis
CTARENPAYGRHMQDAEMFTNAAYMALINWDRFDVFCTLGATSGYLKGNSASFNLVGLFGDNENQSTVKKDAVPNMSFDQSVVELYTDTTFAWSVGARA

SEQ ID NO: 62 - protein, 99 aa, Chlamydia trachomatis
LVERTNPAYGKHMQDAEMFTNCAYTALINWDRFDVFCTLGATSGYLKGNSASFNLVGLFGDGVNATKPAADSIPNVQLNQSVVELYTDTTFAWSVGARA

SEQ ID NO: 63 - protein, 100 aa, Chlamydia trachomatis
NVARPNPAYGKHMQDAEMFTNAAYMALINWDRFDVFCTLGATTGYLKGNSASFNLVGLFGTKTQSSSFNTAKLIPNTALDQSVVELYINTTFAWSVGARA

SEQ ID NO: 64 - protein, 100 aa, Chlamydia trachomatis
NVARPNPAYGKHMQDAEMFTNAAYMALINWDRFDVFCTLGATTGYLKGNSASFNLVGLFGTKTQSSGFDTANIVPNTALNQAVVELYTDTTFAWSVGARA

SEQ ID NO: 65 - protein, 100 aa, Chlamydia trachomatis
NVARPNPAYGKHMQDAEMFTNAAYMALINWDRFDVFCTLGATTGYLKGNSASFNLVGLFGTKTKSSDFNTAKLVPNIALNRAVVELYTDTTFAWSVGARA

SEQ ID NO: 66 - protein, 100 aa, Chlamydia trachomatis
NVARPNPAYGKHMQDAEMFTNAAYMALINWDRFDVFCTLGATTGYLKGNSASFNLVGLFGTKTQSTNFNTAKLVPNTALNQAVVELYTDTTFAWSVGARA

SEQ ID NO: 67 - protein, 96 aa, Chlamydia trachomatis
ASRENPAYGKHMQDAEMFTNAAYMALINWDRFDVFCTLGATSGYLKGNSAAFNLVGLFGRDETAVAADDIPNVSLSQAVVELYTDTAFAWSVGARA

SEQ ID NO: 68 - protein, 100 aa, Chlamydia pneumoniae
YTTAVDRPNPAYNKHLHDAEWFTNAGIFALINWDRFDVFCTLGASNGIRKGNSTAFNLVGLFGVKGTTVNANELPNVSLSNGVVELYTDTSFSWSVGARA

SEQ ID NO: 69 - protein, 100 aa, Chlamydia trachomatis
ALWECGCATLGASFQYAQSKPKVEELNVLCNAAEFTINKPKGYVGQEFPLDLKAGTDGVTGTKDASIDYHEWQASLALSYRLNMFTPYIGVKWSRASFDA

SEQ ID NO: 70 - protein, 100 aa, Chlamydia trachomatis
ALWECGCATLGASFQYAQSKPKVEELNVLCNAAEFTINKPKGYVGKELPLDLTAGTDAATGTKDASIDYHEWQASLALSYRLNMFTPYIGVKWSRASFDA

SEQ ID NO: 71 - protein, 100 aa, Chlamydia trachomatis
ALWECGCATLGASFQYAQSKPKVEELNVLCNAAEFTINKPKGYVGKEFPLDLTAGTDAATGTKDASIDYHEWQASLALSYRLNMFTPYIGVKWSRASFDA

SEQ ID NO: 72 - protein, 100 aa, Chlamydia trachomatis
ALWECGCATLGASFQYAQSKPKVEELNVLCNAAEFTINKPKGYVGQEFPLALIAGTDAATGTKDASIDYHEWQASLALSYRLNMFTPYIGVKWSRASFDA

SEQ ID NO: 73 - protein, 100 aa, Chlamydia trachomatis; identical to SEQ ID NO: 71

SEQ ID NO: 74 - protein, 100 aa, Chlamydia trachomatis
ALWECGCATLGASFQYAQSKPKIEELNVLCNAAEFTINKPKGYVGKEFPLDLTAGTDAATGTKDASIDYHEWQASLSLSYRLNMFTPYIGVKWSRASFDS

SEQ ID NO: 75 - protein, 100 aa, Chlamydia trachomatis
ALWECGCATLGASFQYAQSKPKVEELNVLCNASEFTINKPKGYVGAEFPLNITAGTEAATGTKDASIDYHEWQASLALSYRLNMFTPYIGVKWSRVSFDA

SEQ ID NO: 76 - protein, 100 aa, Chlamydia trachomatis
ALWECGCATLGASFQYAQSKPKVEELNVLCNASEFTINKPKGYVGAEFPLDITAGTEAATGTKDASIDYHEWQASLALSYRLNMFTPYIGVKWSRVSFDA

SEQ ID NO: 77 - protein, 100 aa, Chlamydia trachomatis; identical to SEQ ID NO: 76

SEQ ID NO: 78 - protein, 100 aa, Chlamydia trachomatis
ALWECGCATLGASFQYAQSKPKVEELNVLCDASEFTINKPKGYVGAEFPLDITAGTEAATGTKDASIDYHEWQASLALSYRLNMFTPYIGVKWSRVSFDA

SEQ ID NO: 79 - protein, 100 aa, Chlamydia trachomatis
ALWECGCATLGASFQYAQSKPKVEELNVLCNAAEFTINKPKGYVGQEFPLNIKAGTVSATDTKDASIDYHEWQASLALSYRLNMFTPYIGVKWSRASFDA

SEQ ID NO: 80 - protein, 100 aa, Chlamydia pneumoniae
GLWECGCATLGESFQYAQSKPKVEELNVICNVSQFSVNKPKGYKGVAFPLPTDAGVATATGTKSATINYHEWQVGASLSYRLNSLVPYIGVQWSRATFDA

SEQ ID NO: 81 - protein, 94 aa, Chlamydia trachomatis
DTIRIAQPKSATTVFDVTTLNPTIAGAGDVKASAEGQLGDTMQIVSLQLNKMKSRKSCGIAVGTTIVDADKYAVTVETRLIDERAAHVNAQFRF

SEQ ID NO: 82 - protein, 92 aa, Chlamydia trachomatis
DTIRIAQPKSAETIFDVTTLNPTIAGAGDVKTSAEGQLGDTMQIVSLQLNMKSRKCGIAVGTTIVDADKYAITVETRLIDERAAHVNAQFRF

SEQ ID NO: 83 - protein, 94 aa, Chlamydia trachomatis
DTIRIAQPKSATAIFDTTTLNPTIAGAGDVKTGTEGQLGDTMQIVSLQLNKMKSRKSCGIAVGTTIVDADKYAVTVETRLIDERAAHVNAQFRF

SEQ ID NO: 84 - protein, 94 aa, Chlamydia trachomatis
DTIRIAQPKSATAIFDTTTLNPTIAGAGDVKASAEGQLGDTMQIVSLQLNKMKSRKSCGIAVGTTIVDADKYAVTVETRLIDERAAHVNAQFRF

SEQ ID NO: 85 - protein, 94 aa, Chlamydia trachomatis
DTIRIAQPKLATAIFDTTTLNPTIAGAGDEKANAEGQLGDTMQIVSLQLNKMKSRKSCGIAVGTTIVDADKYAVTVETRLIDERAAHVNAQFRF

SEQ ID NO: 86 - protein, 95 aa, Chlamydia trachomatis
DTIRIAQPRLVTPVVDITTLNPTIAGACDSKAGNTEGQISDTMQIVSLQLNKMKSRKSCGIAVGTTIVDADKYAVTVETRLIDERAAHVNAQFRF

SEQ ID NO: 87 - protein, 95 aa, Chlamydia trachomatis
DTIRIAQPKLAEAILDVTTLNRTTAGKGSVVSAGTDNELADTMQIVSLQLNKMKSRKSCGIAVGTTIVDADKYAVTVEARLIDERAAHVNAQFRF

SEQ ID NO: 88 - protein, 94 aa, Chlamydia trachomatis
DTIRIAQPKLAKPVLDTTTLNPTIAGKGTVVSSAENELADTMQIVSLQLNKMKSRKSCGIAVGTTIVDADKYAVTVETRLIDERAAHVNAQFRF

SEQ ID NO: 89 - protein, 94 aa, Chlamydia trachomatis
DTIRIAQPKLAEAILDVTTLNPTIAGKGTVVASGSDNDLADTMQIVSLQLNKMKSRKSCGIAVGTTIVDADKYAVTVETRLIDERAAHVNAQFR

SEQ ID NO: 90 - protein, 95 aa, Chlamydia trachomatis
DTIRIAQPKLAEAVLDVTTLNPTIAGKGSVVASGSENELADTMQIVSLQLNKMKSRKSCGIAVGTTIVDADKYAVTVETRLIDERAAHVNAQFRF

SEQ ID NO: 91 - protein, 91 aa, Chlamydia trachomatis
DTIRIAQPKLETSILKMTTWNPTISGSGIDVDTKITDTLQIVSLQLNKMKSRKSCLIAIGTTIVDADKYAVTVETRLIDERAAHVNAQFRF

SEQ ID NO: 92 - protein, 93 aa, Chlamydia pneumoniae
DNIRIAQPKLPTAVLNLTAWNPSLLGNATALSTTDSFSDFMQIVSCQINKFKSRKACVTAVATLIVDADKWSLTAEARLNDERAAHSGAQFRF

SEQ ID NO: 93 - protein, 17 aa, Chlamydia pneumoniae
CTGSAAANYTTAVDRPN

SEQ ID NO: 94 - protein, 18 aa, Chlamydia trachomatis
CTGDADLTTAPTPASREN

SEQ ID NO: 95 - protein, 19 aa, Chlamydia trachomatis
CTTATGNAAAPSTCTAREN

SEQ ID NO: 96 - protein, 17 aa, Chlamydia psitacci
CASGTASNTTVAADRSN

SEQ ID NO: 97 - protein, 15 aa, Chlamydia pneumoniae
CFGVKGTTVNANELP

SEQ ID NO: 98 - protein, 15 aa, Chlamydia trachomatis
CFGRDETAVAADDIP

SEQ ID NO: 99 - protein, 18 aa, Chlamydia trachomatis
CFGDNENHATVSDSKLVP

SEQ ID NO: 100 - protein, 14 aa, Chlamydia psitacci
CIGLAGTDFANQRP

SEQ ID NO: 101 - protein, 13 aa, Chlamydia pneumoniae
CQINKFKSRKACG

SEQ ID NO: 102 - protein, 13 aa, Chlamydia trachomatis
CQINKMKSRFACG

SEQ ID NO: 103 - protein, 13 aa, Chlamydia trachomatis
CQLNKMKSRKACG

SEQ ID NO: 104 - protein, 13 aa, Chlamydia psitacci
CQINKFKSRFACG

SEQ ID NO: 105 - protein, 6 aa, Chlamydia pneumoniae
RKKERS

SEQ ID NO: 106 - protein, 10 aa, Chlamydia pneumoniae
STECNSQSPQ

SEQ ID NO: 107 - protein, 13 aa, Ebola virus
NPNLHYWTTQDEG

SEQ ID NO: 108 - protein, 22 aa, Ebola virus
SGQSPARTSSDPGTNTTTEDHK

SEQ ID NO: 109 - protein, 9 aa, Ebola virus
TGGRRTRRE

SEQ ID NO: 110 - protein, 10 aa, Ebola virus
RDRFKRTSFF

SEQ ID NO: 111 - protein, 12 aa, Ebola virus
EQHHRRTDNDST

SEQ ID NO: 112 - protein, 11 aa, Ebola virus
ENTNTSKSTDF

SEQ ID NO: 113 - protein, 12 aa, Ebola virus
YTSGKRSNTTGK

SEQ ID NO: 114 - protein, 11 aa, Ebola virus
TTTSPQNHSET

SEQ ID NO: 115 - protein, 11 aa, Ebola virus
PDQGDNDNWWT

SEQ ID NO: 116 - protein, 12 aa, Ebola virus
TISTSPQSLTTK

SEQ ID NO: 117 - protein, 16 aa, Ebola virus
TEDPSSGYYSTTIRYQ

SEQ ID NO: 118 - protein, 15 aa, Ebola virus
THHQDTGEESASSGK

(19) United States
(12) Patent Application Publication
(10) Pub. No.: US 2006/0073531 A1
(43) Pub. Date: Apr. 6, 2006

(54) Anti-chlamydial antibodies and uses thereof

(75) Inventors: William M. Mitchell, Nashville, TN (US); Charles W. Stratton, Nashville, TN (US)

Correspondence Address: CLARK & ELBING LLP, 101 FEDERAL STREET, BOSTON, MA, 02110, US

(73) Assignee: Vanderbilt University, Nashville, TN (US)

(21) Appl. No.: 11/252,479

(22) Filed: Oct. 18, 2005

Related U.S. Application Data
(63) Continuation-in-part of application No. 10/020,269, filed on Dec. 14, 2001, now abandoned.
Application No. 10/020,269 is a continuation of application No. 09/025,596, filed on Feb. 18, 1998, now Pat. No. 6,340,463.
Application No. 09/025,596 is a continuation-in-part of application No. 08/911,593, filed on Aug. 14, 1997, now abandoned.
(63) Continuation-in-part of application No. 10/873,768, filed on Jun. 22, 2004, now abandoned.
Application No. 10/873,768 is a continuation of application No. 09/709,201, filed on Nov. 8, 2000, now Pat. No. 6,838,552.
Application No. 09/709,201 is a continuation of application No. 09/025,521, filed on Feb. 18, 1998, now abandoned.
Application No. 09/025,521 is a continuation-in-part of application No. 08/911,593, filed on Aug. 14, 1997, now abandoned.
(60) Provisional application No. 60/023,921, filed on Aug. 14, 1996.

(51) Int. Cl. G01N 33/554; G01N 33/569; C07K 16/12

(52) U.S. Cl. 435/7.32; 530/388.4

(57) ABSTRACT

The invention relates to anti-chlamydial antibodies and uses of anti-chlamydial antibodies for detection of the presence of chlamydia.

### CROSS-REFERENCE TO RELATED APPLICATIONS

This application is a continuation-in-part of U.S. patent application Ser. No. 10/020,269, filed Dec. 14, 2001, which is a continuation of U.S. patent application Ser. No. 09/025,596, filed Feb. 18, 1998, which is a continuation-in-part of U.S. patent application Ser. No. 08/911,593, filed Aug. 14, 1997. This application is also a continuation-in-part of U.S. patent application Ser. No. 10/873,768, filed Jun. 22, 2004, which is a continuation-in-part of U.S. patent application Ser. No. 08/911,593. U.S. patent application Ser. No. 08/911,593 claims benefit from U.S. provisional application U.S. Pat. Ser. No. 60/023,921, filed Aug. 14, 1996. The entire teachings of each of the foregoing applications are incorporated herein by reference.

### BACKGROUND OF THE INVENTION

Antibodies are widely used in diagnostic assays in both human and veterinary medicine. Uses include enzyme-linked immunosorbent analysis (ELISA), quantitative antigen capture analysis, radioisotope-tagged reagents for in vivo localization of target antigens, and for in vivo localization of cytotoxic agents to target cells (i.e., immunotoxic therapy). The minimum epitope size for protein antigens is generally considered to be 5-6 amino acids, either as a linear sequence or as non-contiguous amino acids whose spatial placement defines the epitope (i.e., conformational epitope). Specificity is provided by the large number of potential amino acid epitopic sequences possible for a minimum epitope (i.e., 5²⁰).

Most commonly, large antigens or microbial organisms are used to induce antibody responses in order to insure the presentation of good antigenic sequences in the host animal. The use of these multivalent antigens for the production of polyclonal antibodies generally requires host-based adsorption of the sera to reduce non-specific cross-reactive antibody species. Monoclonal antibodies avoid this pitfall but frequently result in reagents whose specific epitopic specificity is unknown.

### SUMMARY OF THE INVENTION

The invention relates to a method of identifying an antigenic amino acid subsequence from within a larger amino acid sequence comprising the steps of evaluating the hydrophilicity of subsequences of an amino acid sequence of interest; evaluating the flexibility of subsequences of the amino acid sequence of interest; and selecting an amino acid subsequence having overlapping regions of hydrophilicity and flexibility. In particular embodiments, the larger amino acid sequence is selected from the group consisting of polypeptides expressed by members of the Chlamydia genus.

The invention also relates to antigenic amino acid subsequences identified by the methods described herein. In particular embodiments, the invention pertains to an antigenic amino acid subsequence selected from the group consisting of SEQ ID NOS: 1-118.

The invention also relates to antibodies which are specific for the antigenic amino acid subsequences described herein. For example, the invention pertains to monoclonal antibodies specific for antigenic amino acid subsequences described herein.

The invention also relates to diagnostic and therapeutic methods utilizing the described antigenic amino acid subsequences and antibodies thereto.

The invention also encompasses a method for evaluating the infection status of an individual and/or the progress of therapy in an individual undergoing therapy for infection caused by Chlamydia. The method comprises quantifying antibody titer or other measure to the pathogen and comparing the measure to antibody measure quantified at a time earlier in the therapy, whereby the difference between the measures is indicative of the progress of the therapy. The invention also pertains to a method for monitoring the course of therapy for treating infection by Chlamydia, comprising determining presence or absence of Chlamydia in an infected individual at time intervals during course of therapy. In a particular embodiment, this is determined by PCR assay or antigen capture assay for pathogen DNA.

Detection of the presence of Chlamydia in a sample of biological material taken from an individual thought to be infected therewith is important in determining the course of therapy and the agents to be used. This can be achieved by detecting the presence of DNA encoding MOMP of Chlamydia or other chlamydial genes in the individual. In one aspect of the invention, diseases associated with Chlamydia infection, such as inflammatory diseases, autoimmune diseases and diseases in which the individual is immunocompromised, can be treated by managing (e.g., significantly reducing infection or eradicating) the Chlamydia infection using the novel approach described herein. Both clinical and serological improvements/resolutions in patient status have been demonstrated.

### BRIEF DESCRIPTION OF THE DRAWINGS

FIGS. 1A and 1B are sequence alignments of various Chlamydia MOMPs. Variable domains (VD1-VD4) are boxed. Sequences are aligned with the L2 serovar of C. trachomatis and are ranked from highest homology (B, D, E, L1) to lower homology (F, C, and A, H, L3). MU is the mouse pneumonitis C. trachomatis. PN refers to the human C. pneumonia. Deletions are indicated by (−). A blank indicates the same residue as L2. The leader sequence is bracketed. Underlined seven residue segments are predicted to contain the most flexible peptide backbone based on the L2 sequence. Asterisks indicate the most hydrophilic region.

FIG. 2 illustrates the predicted antigenic sequences from variable domains 1 (VD1) of various Chlamydia species. The boxed cysteine (C) residue is not part of the native sequence but has been added at the amino terminus for cross-linking to carrier proteins used in immunization.

FIG. 3 illustrates the predicted antigenic sequences from variable domain 2 (VD2) of various Chlamydia species. The boxed cysteine (C) residue is not part of the native sequence but has been added at the amino terminus for cross-linking to carrier proteins used in immunization.

FIG. 4 illustrates the predicted antigenic sequences from a common domain of various Chlamydia species. The shaded box indicates hydrophilic mobile region common to each with expected cross-reactivity for antibodies specific for the sequence. The boxed cysteine (C) residue is not part of the native sequence but has been added at the amino terminus for cross-linking to carrier proteins used in immunization.

### DETAILED DESCRIPTION OF THE INVENTION

Globular proteins have a hydrophobic core, with the external surfaces bearing relatively hydrophilic sequences. It is these segments in native proteins which are most likely to be recognized by antibodies. Work described herein describes methods for identifying linear amino acid antigenic sequences for the production of both polyclonal and monoclonal antibodies to defined antigenic domains. One significant advantage of this technique is that it provides antibodies to a known epitope of a target antigen or organism.

The identification of antigenic domains described herein is based on the overlap of the most hydrophilic peptide segments of an antigen with those peptide segments with a concomitant predicted peptide flexibility. Increased flexibility allows more conformational degrees of freedom for optimal fit into an antibody binding site. Aromatic amino acids are frequently found in antigenic epitopes although hydrophobic with bulky R groups. This decrease in the relative hydrophobicity and flexibility of the peptide sequence containing the aromatic residue is compensated for if accessible (i.e., surface of the antigen).

The relative hydrophilicity of peptide domains is based on the individual hydrophilicity of each amino acid in six or more residue segments as defined by Hopp and Woods (Hopp and Woods, Proceedings of National Academy of Sciences USA 78:3824-3828). Flexibility of the peptide chain at each Cα residue is measured from the average value of the atomic temperature factor as affected by adjacent residues. Amino acids which result in rigidity of the chain include alanine, valine, leucine, isoleucine, tyrosine, phenylalanine, tryptophan, cysteine, methionine, and histidine. Flexibility is computed by averaging the rigidity factor (B value) along a seven residue segment using the following expression (Karplus and Shulz, Naturwissenschaften 72:212-213 (1985); Van Regenmortel, Trends in Biochemical Sciences (TIBS) 12:36-39 (1986)):
F=Bᵢ+0.75(Bᵢ₋₁+Bᵢ₊₁)+0.5(Bᵢ₋₂+Bᵢ₊₂)+0.25(Bᵢ₋₃+Bᵢ₊₃)

With respect to identification of larger proteins or polypeptides from which the antigenic amino acid subsequences are selected, bands identified by gel analysis can be isolated and purified by HPLC, and the resulting purified protein can be sequenced. Alternatively, the purified protein can be enzymatically digested by methods known in the art to produce polypeptide fragments which can be sequenced. The sequencing can be performed, for example, by the methods of Wilm et al. (Nature 379:466-469 (1996)). The protein can be isolated by conventional means of protein biochemistry and purification to obtain a substantially pure product, i.e., 80, 95 or 99% free of cell component contaminants, as described in Jacoby, Methods in Enzymology Volume 104, Academic Press, New York (1984); Scopes, Protein Purification, Principles and Practice, 2nd Edition, Springer-Verlag, New York (1987); and Deutscher (ed), Guide to Protein Purification, Methods in Enzymology, Vol. 182 (1990). If the protein is secreted, it can be isolated from the supernatant in which the host cell is grown. If not secreted, the protein can be isolated from a lysate of the host cells.

In addition to substantially full-length polypeptides used as the source of the selected antigenic amino acid subsequences, biologically active fragments of polypeptides, or analogs thereof, including organic molecules which simulate the interactions of the polypeptides, can be used. Biologically active fragments include any portion of the full-length polypeptide which has a biological function, including ligand binding, and antibody binding. Ligand binding includes binding by nucleic acids, proteins or polypeptides, small biologically active molecules, or large cellular structures. Amino acid sequences identified as antigenic from de novo sequence determination of cDNA reading frames or the isolated protein of interest, or by established sequences from GeneBank and the (PDB), can be most conveniently synthesized by solid phase peptide synthesis using either standard F-Monc or t-Boc methodologies.

This invention also pertains to an isolated polypeptide comprising the antigenic amino acid subsequences of the invention. The encoded proteins or polypeptides of the invention can be partially or substantially purified (e.g., purified to homogeneity), and/or are substantially free of other proteins. According to the invention, the amino acid sequence of the polypeptide can be that of the naturally-occurring polypeptide or can comprise alterations therein. Such alterations include conservative or non-conservative amino acid substitutions, additions and deletions of one or more amino acids; however, such alterations should preserve at least one activity of the encoded protein or polypeptide, i.e., the altered or mutant protein should be an active derivative of the naturally-occurring protein. For example, the mutation(s) can preferably preserve the three dimensional configuration of the binding and/or catalytic site of the native protein, the hydrophilicity and/or flexibility of the polypeptide. The presence or absence of biological activity or activities can be determined by various functional assays as described herein. Moreover, amino acids which are essential for antigenicity or the function of the encoded protein or polypeptide can be identified by methods known in the art. Particularly useful methods include identification of conserved amino acids in the family or subfamily, site-directed mutagenesis and alanine-scanning mutagenesis (for example, Cunningham and Wells, Science 244:1081-1085 (1989)), crystallization and nuclear magnetic resonance. The altered polypeptides produced by these methods can be tested for particular biologic activities, including immunogenicity and antigenicity, as described herein.

Specifically, appropriate amino acid alterations can be made on the basis of several criteria, including hydrophobicity, hydrophilicity, basic or acidic character, charge, polarity, size, the presence or absence of a functional group (e.g., —SH or a glycosylation site), rigidity or flexibility, and aromatic character. Assignment of various amino acids to similar groups based on the properties above will be readily apparent to the skilled artisan; further appropriate amino acid changes can also be found in Bowie et al. (Science 247:1306-1310 (1990)).

Polypeptides of the invention can also be a fusion protein comprising all or a portion of the amino acid sequence fused to an additional component. Additional components, such as radioisotopes and antigenic tags, can be selected to assist in the isolation or purification of the polypeptide or to extend the half life of the polypeptide; for example, a hexahistidine tag would permit ready purification by nickel chromatography. Polypeptides or amino acid sequences described herein can be isolated from naturally-occurring sources, chemically synthesized or recombinantly produced by methods known in the art.

The present invention also relates to nucleotide sequences (nucleic acid molecules) which encode the antigenic amino acid subsequences or polypeptides of the invention. As appropriate, nucleic acid molecules of the present invention can be RNA, for example, mRNA, or DNA, such as cDNA and genomic DNA. DNA molecules can be double-stranded or single-stranded; single stranded RNA or DNA can be either the coding, or sense, strand or the non-coding, or antisense, strand. The nucleic acid molecule can include all or a portion of the coding sequence of a gene and can further comprise additional non-coding sequences such as introns and non-coding 3′ and 5′ sequences (including regulatory sequences, for example). Additionally, the nucleic acid molecule can be fused to a marker sequence, for example, a sequence which encodes a polypeptide to assist in isolation or purification of the polypeptide. Such sequences include, but are not limited to, those which encode a glutathione-S-transferase (GST) fusion protein and those which encode a hemaglutin A (HA) polypeptide marker from influenza.

As used herein, an “isolated” gene or nucleic acid molecule is intended to mean a gene or nucleic acid molecule which is not flanked by nucleic acid molecules which normally (in nature) flank the gene or nucleic acid molecule (such as in genomic sequences) and/or has been completely or partially purified from other transcribed sequences (as in a cDNA or RNA library). For example, an isolated nucleic acid of the invention may be substantially isolated with respect to the complex cellular milieu in which it naturally occurs. In some instances, the isolated material will form part of a composition (for example, a crude extract containing other substances), buffer system or reagent mix. In other circumstance, the material may be purified to essential homogeneity, for example as determined by PAGE or column chromatography such as HPLC. Preferably, an isolated nucleic acid comprises at least about 50, 80 or 90 percent (on a molar basis) of all macromolecular species present. Thus, an isolated gene or nucleic acid molecule can include a gene or nucleic acid molecule which is synthesized chemically or by recombinant means. Recombinant DNA contained in a vector are included in the definition of “isolated” as used herein. Also, isolated nucleic acid molecules include recombinant DNA molecules in heterologous host cells, as well as partially or substantially purified DNA molecules in solution. In vivo and in vitro RNA transcripts of the DNA molecules of the present invention are also encompassed by “isolated” nucleic acid molecules. Such isolated nucleic acid molecules are useful in the manufacture of the encoded polypeptide, as probes for isolating homologous sequences (e.g., from other mammalian species), for gene mapping (e.g., by in situ hybridization with chromosomes), or for detecting expression of the gene in tissue (e.g., human tissue such as liver tissue), such as by Northern blot analysis.

The invention also pertains to nucleic acid molecules which hybridize under high stringency hybridization conditions (e.g., for selective hybridization) to a nucleotide sequence described herein. Hybridization probes are oligonucleotides which bind in a base-specific manner to a complementary strand of nucleic acid. Appropriate stringency conditions are known to those skilled in the art or can be found in standard texts such as Current Protocols in Molecular Biology, John Wiley & Sons, N.Y. (1989), 6.3.1-6.3.6. For example, stringent hybridization conditions include a salt concentration of no more than 1 M and a temperature of at least 25° C. In one embodiment, conditions of 5×SSPE (750 mM NaCl, 50 mM NaPhosphate, 5 mM EDTA, pH 7.4) and a temperature of 25-30° C., or equivalent conditions, are suitable for specific probe hybridizations. Equivalent conditions can be determined by varying one or more of the parameters given as an example, as known in the art, while maintaining a similar degree of identity or similarity between the target nucleic acid molecule and the primer or probe used. Hybridizable nucleic acid molecules are useful as probes and primers for diagnostic applications.

Accordingly, the invention pertains to nucleic acid molecules which have a substantial identity with the nucleic acid molecules described herein and which encode antigenic amino acid sequences; particularly preferred are nucleic acid molecules which have at least about 90%, and more preferably at least about 95% identity with nucleic acid molecules described herein. Thus, DNA molecules which comprise a sequence which is different from the naturally-occurring nucleic acid molecule but which, due to the degeneracy of the genetic code, encode the same polypeptide are the subject of this invention. The invention also encompasses variations of the nucleic acid molecules of the invention, such as those encoding portions, analogues or derivatives of the encoded polypeptide. Such variations can be naturally-occurring, such as in the case of allelic variation, or non-naturally-occurring, such as those induced by various mutagens and mutagenic processes. Intended variations include, but are not limited to, addition, deletion and substitution of one or more nucleotides which can result in conservative or non-conservative amino acid changes, including additions and deletions. Preferably, the nucleotide or amino acid variations are silent; that is, they do not alter the characteristics or activity of the encoded protein or polypeptide. As used herein, activities of the encoded protein or polypeptide include, but are not limited to, catalytic activity, binding function, antigenic function and oligomerization function.

The nucleotide sequences described herein can be amplified as needed by methods known in the art. For example, this can be accomplished by e.g., PCR. See generally PCR Technology: Principles and Applications for DNA Amplification (ed. H. A. Erlich, Freeman Press, NY, N.Y., 1992); PCR Protocols: A Guide to Methods and Applications (eds. Innis, et al., Academic Press, San Diego, Calif., 1990); Mattila et al., Nucleic Acids Res. 19, 4967 (1991); Eckert et al., PCR Methods and Applications 1, 17 (1991); PCR (eds. McPherson et al., IRL Press, Oxford); and U.S. Pat. No. 4,683,202.

Other suitable amplification methods include the ligase chain reaction (LCR) (see Wu and Wallace, Genomics 4, 560 (1989), Landegren et al., Science 241, 1077 (1988), transcription amplification (Kwoh et al., Proc. Natl. Acad. Sci. USA 86, 1173 (1989)), and self-sustained sequence replication (Guatelli et al., Proc. Nat. Acad. Sci. USA, 87, 1874 (1990)) and nucleic acid based sequence amplification (NASBA). The latter two amplification methods involve isothermal reactions based on isothermal transcription, which produce both single stranded RNA (ssRNA) and double stranded DNA (dsDNA) as the amplification products in a ratio of about 30 or 100 to 1, respectively.

The amplified DNA can be radiolabelled and used as a probe for screening a cDNA library. Corresponding clones can be isolated, DNA can obtained following in vivo excision, and the cloned insert can be sequenced in either or both orientations by art recognized methods, to identify the correct reading frame encoding a protein of the appropriate molecular weight. For example, the direct analysis of the nucleotide sequence of nucleic acid molecules of the present invention can be accomplished using either the dideoxy chain termination method or the Maxam Gilbert method (see Sambrook et al., Molecular Cloning, A Laboratory Manual (2nd Ed., CSHP, New York 1989); Zyskind et al., Recombinant DNA Laboratory Manual, (Acad. Press, 1988)). Using these or similar methods, the polypeptide(s) and the DNA encoding the polypeptide can be isolated, sequenced and further characterized.

The invention also provides expression vectors containing a nucleic acid sequence described herein, operably linked to at least one regulatory sequence. Many such vectors are commercially available, and other suitable vectors can be readily prepared by the skilled artisan. “Operably linked” is intended to mean that the nucleic acid molecule is linked to a regulatory sequence in a manner which allows expression of the nucleic acid sequence. Regulatory sequences are art-recognized and are selected to produce the encoded polypeptide. Accordingly, the term “regulatory sequence” includes promoters, enhancers, and other expression control elements which are described in Goeddel, Gene Expression Technology: Methods in Enzymology 185, Academic Press, San Diego, Calif. (1990). For example, the native regulatory sequences or regulatory sequences native to the transformed host cell can be employed. It should be understood that the design of the expression vector may depend on such factors as the choice of the host cell to be transformed and/or the type of polypeptide desired to be expressed. For instance, the polypeptides of the present invention can be produced by ligating the cloned gene, or a portion thereof, into a vector suitable for expression in either prokaryotic cells, eukaryotic cells or both (see, for example, Broach, et al., Experimental Manipulation of Gene Expression, ed. M. Inouye (Academic Press, 1983) p. 83; Molecular Cloning: A Laboratory Manual, 2nd Ed., ed. Sambrook et al. (Cold Spring Harbor Laboratory Press, 1989) Chapters 16 and 17). Typically, expression constructs will contain one or more selectable markers, including, but not limited to, the gene that encodes dihydrofolate reductase and the genes that confer resistance to neomycin, tetracycline, ampicillin, chloramphenicol, kanamycin and streptomycin resistance.

Prokaryotic and eukaryotic host cells transfected by the described vectors are also provided by this invention. For instance, cells which can be transfected with the vectors of the present invention include, but are not limited to, bacterial cells such as E. coli (e.g., E. coli K12 strains, Streptomyces, Pseudomonas, Serratia marcescens and Salmonella typhimurium, insect cells (baculovirus), including Drosophila, fungal cells, such as yeast cells, plant cells and mammalian cells, such as thymocytes, Chinese hamster ovary cells (CHO), and COS cells.

Thus, a nucleic acid molecule described herein can be used to produce a recombinant form of the polypeptide via microbial or eukaryotic cellular processes. Ligating the polynucleic acid molecule into a gene construct, such as an expression vector, and transforming or transfecting into hosts, either eukaryotic (yeast, avian, insect, plant or mammalian) or prokaryotic (bacterial cells), are standard procedures used in producing other well known proteins. Similar procedures, or modifications thereof, can be employed to prepare recombinant polypeptides according to the present invention by microbial means or tissue-culture technology. Accordingly, the invention pertains to the production of encoded polypeptides by recombinant technology.

The polypeptides of the present invention can be isolated or purified (e.g., to homogeneity) from recombinant cell culture by a variety of processes. These include, but are not limited to, anion or cation exchange chromatography, ethanol precipitation, affinity chromatography and high performance liquid chromatography (HPLC). The particular method used will depend upon the properties of the polypeptide and the selection of the host cell; appropriate methods will be readily apparent to those skilled in the art.

The present invention also relates to antibodies which bind an antigenic amino acid sequence or subsequence of the invention. For instance, polyclonal and monoclonal antibodies, including non-human and human antibodies, humanized antibodies, chimeric antibodies and antigen-binding fragments thereof (Current Protocols in Immunology, John Wiley & Sons, N.Y. (1994); EP Application 173,494 (Morrison); International Patent Application WO86/01533 (Neuberger); and U.S. Pat. No. 5,225,539 (Winters)) which bind to the described amino acid sequence or subsequence are within the scope of the invention. A mammal, such as a mouse, rat, hamster or rabbit, can be immunized with an immunogenic form of the amino acid subsequence. Techniques for conferring immunogenicity on a polypeptide include conjugation to carriers or other techniques well known in the art. The polypeptide can be administered in the presence of an adjuvant. The progress of immunization can be monitored by detection of antibody titers in plasma or serum. Standard ELISA or other immunoassays can be used with the immunogen as antigen to assess the levels of antibody.

Following immunization, anti-peptide antisera can be obtained, and if desired, polyclonal antibodies can be isolated from the serum. Monoclonal antibodies can also be produced by standard techniques which are well known in the art (Kohler and Milstein, Nature 256:495-497 (1975); Kozbar et al., Immunology Today 4:72 (1983); and Cole et al., Monoclonal Antibodies and Cancer Therapy, Alan R. Liss, Inc., pp. 77-96 (1985)). The term “antibody” as used herein is intended to include fragments thereof, such as Fab and F(ab)₂ and antigen binding fragments. Antibodies described herein can be used to inhibit the activity of the polypeptides and proteins described herein, particularly in vitro and in cell extracts, using methods known in the art.

Additionally, such antibodies, in conjunction with a label, such as a radioactive label, can be used to assay for the presence of the expressed protein in a cell from, e.g., a tissue sample, and can be used in an immunoabsorption process, such as an ELISA, to isolate the protein or polypeptide. Tissue samples which can be assayed include human tissues, e.g., differentiated and non-differentiated cells. Examples include bone marrow, thymus, kidney, liver, brain, pancreas, fibroblasts and epithelium. These antibodies are useful in diagnostic assays, or as an active ingredient in a pharmaceutical composition.

The invention also relates to immunogenic compositions comprising amino acid sequences described herein, as well as vaccine compositions comprising polypeptides or antibodies described herein. Peptides and antibodies identified by methods described herein can also be used in a variety of assay and protein processing applications, including, but not limited to, radioimmunoassays, ELISA, antigen capture assays, competitive inhibition assays, affinity chromatography, Western Blotting, Labeled-antibody assays such as immunofluorescence assays, immunohistochemical staining assays and immunoprecipitation assays. The antibodies, alone or linked to particular toxins, can also be used for a variety of therapeutic and other purposes, including removing specific lymphocyte subsets, inhibiting cell function, inhibiting graft rejection, alleviating or suppressing autoimmune disease, and attaching to tumors.

The present invention also pertains to pharmaceutical compositions comprising antigenic amino acid sequences or subsequences and other antibodies described herein. For instance, a composition of the present invention can be formulated with a physiologically acceptable medium to prepare a pharmaceutical composition. The particular physiological medium may include, but is not limited to, water, buffered saline, polyols (e.g., glycerol, propylene glycol, liquid polyethylene glycol) and dextrose solutions. The optimum concentration of the active ingredient(s) in the chosen medium can be determined empirically, according to well known procedures, and will depend on the ultimate pharmaceutical formulation desired. Methods of introduction of exogenous polypeptides at the site of treatment include, but are not limited to, intradermal, intramuscular, intraperitoneal, intravenous, subcutaneous, oral and intranasal. Other suitable methods of introduction can also include gene therapy, rechargeable or biodegradable devices and slow release polymeric devices. The pharmaceutical compositions of this invention can also be administered as part of a combinatorial therapy with other agents.

The nucleic acid sequences described herein can also be used for genetic immunization. The term “genetic immunization,” as used herein, refers to inoculation of a vertebrate, particularly a mammal, with a nucleic acid vaccine directed against a pathogenic agent, such as Chlamydia, resulting in protection of the vertebrate against the pathogenic agent. Representative vertebrates include mice, dogs, cats, chickens, sheep, goats, cows, horses, pigs, non-human primates, and humans. A “nucleic acid vaccine” or “DNA vaccine” as used herein, is a nucleic acid construct comprising a polynucleotide encoding a polypeptide antigen, particularly an antigenic amino acid subsequence identified by methods described herein. The nucleic acid construct can also include transcriptional promoter elements, enhancer elements, splicing signals, termination and polyadenylation signals, and other nucleic acid sequences.

“Protection against the pathogenic agent” as used herein refers to generation of an immune response in the vertebrate, the immune response being protective (partially or totally) against manifestations of the disease caused by the pathogenic agent. A vertebrate that is protected against disease may be infected with the pathogenic agent, but to a lesser degree than would occur without immunization; may be infected with the pathogenic agent, but does not exhibit disease symptoms; or may be infected with the pathogenic agent, but exhibits fewer disease symptoms than would occur without immunization. Alternatively, the vertebrate that is protected against disease may not become infected with the pathogenic agent at all, despite exposure to the agent.

The nucleic acid vaccine can be produced by standard methods. For example, using known methods, a nucleic acid encoding polypeptide antigen of interest, e.g., DNA encoding an antigenic amino acid subsequence, can be inserted into an expression vector to construct a nucleic acid vaccine (see Maniatis et al., Molecular Cloning, A Laboratory Manual, 2nd edition, Cold Spring Harbor Laboratory Press (1989)).

The individual vertebrate is inoculated with the nucleic acid vaccine (i.e., the nucleic acid vaccine is administered), using standard methods. The vertebrate can be inoculated subcutaneously, intravenously, intraperitoneally, intradermally, intramuscularly, topically, orally, rectally, nasally, buccally, vaginally, by inhalation spray, or via an implanted reservoir in dosage formulations containing conventional non-toxic, physiologically acceptable carriers or vehicles. Alternatively, in a preferred embodiment, the vertebrate is inoculated with the nucleic acid vaccine through the use of a particle acceleration instrument (a “gene gun”). The form in which it is administered (e.g., capsule, tablet, solution, emulsion) will depend in part on the route by which it is administered. For example, for mucosal administration, nose drops, inhalants or suppositories can be used.

The nucleic acid vaccine can be administered in conjunction with known adjuvants. The adjuvant is administered in a sufficient amount, which is that amount that is sufficient to generate an enhanced immune response to the nucleic acid vaccine. The adjuvant can be administered prior to (e.g., 1 or more days before) inoculation with the nucleic acid vaccine; concurrently with (e.g., within 24 hours of) inoculation with the nucleic acid vaccine; contemporaneously (simultaneously) with the nucleic acid vaccine (e.g., the adjuvant is mixed with the nucleic acid vaccine, and the mixture is administered to the vertebrate); or after (e.g., 1 or more days after) inoculation with the nucleic acid vaccine. The adjuvant can also be administered at more than one time (e.g., prior to inoculation with the nucleic acid vaccine and also after inoculation with the nucleic acid vaccine). As used herein, the term “in conjunction with” encompasses any time period, including those specifically described herein and combinations of the time periods specifically described herein, during which the adjuvant can be administered so as to generate an enhanced immune response to the nucleic acid vaccine (e.g., an increased antibody titer to the antigen encoded by the nucleic acid vaccine, or an increased antibody titer to the pathogenic agent). The adjuvant and the nucleic acid vaccine can be administered at approximately the same location on the vertebrate; for example, both the adjuvant and the nucleic acid vaccine are administered at a marked site on a limb of the vertebrate.

In a particular embodiment, the nucleic acid construct is co-administered with a transfection-facilitating cationic lipid. In a preferred embodiment, the cationic lipid is dioctylglycylspermine (DOGS) (U.S. patent application Ser. Nos. 08/372,429 and 08/544,575, PCT application Ser. No. PCT/US96/16845 and published PCT application publication no. WO 96/21356). In a particular embodiment, the nucleic acid construct is co-administered with a transfection-facilitating cationic lipid and an amount of 1,25(OH)₂D3 effective to produce a mucosal response. In a preferred embodiment, the nucleic acid construct is complexed with a transfection-facilitating cationic lipid.

The teachings of all references cited herein are specifically incorporated herein by reference. The teachings of copending U.S. patent application Ser. No. 09/025,521, entitled “Diagnosis and Management of Infection Caused by Chlamydia” by William M. Mitchell and Charles W. Stratton, filed concurrently with the present application, are also incorporated herein by reference in their entirety.

### EXAMPLES

Examples of the predictive power of the methodology described herein include the following:

1) Antigenicity of the MOMP (Major Outer Membrane Protein) of Chlamydia

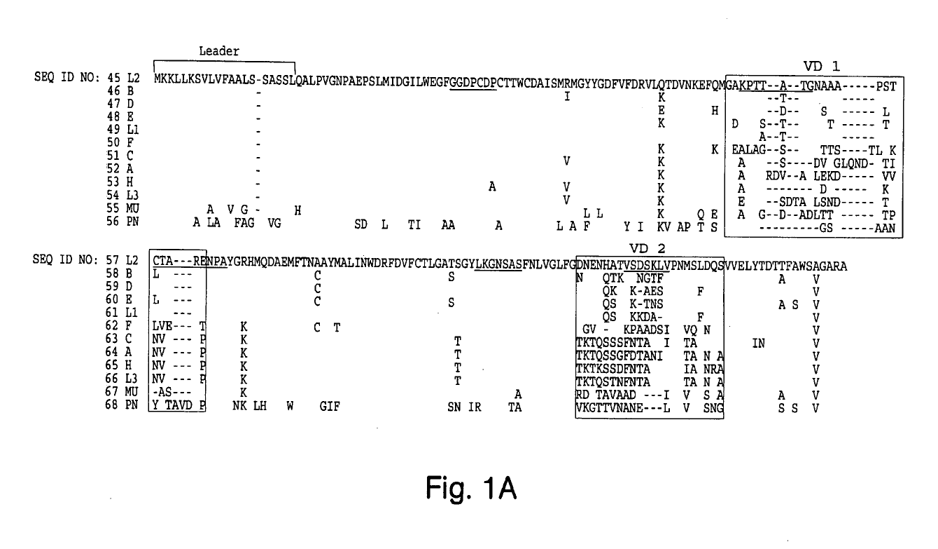

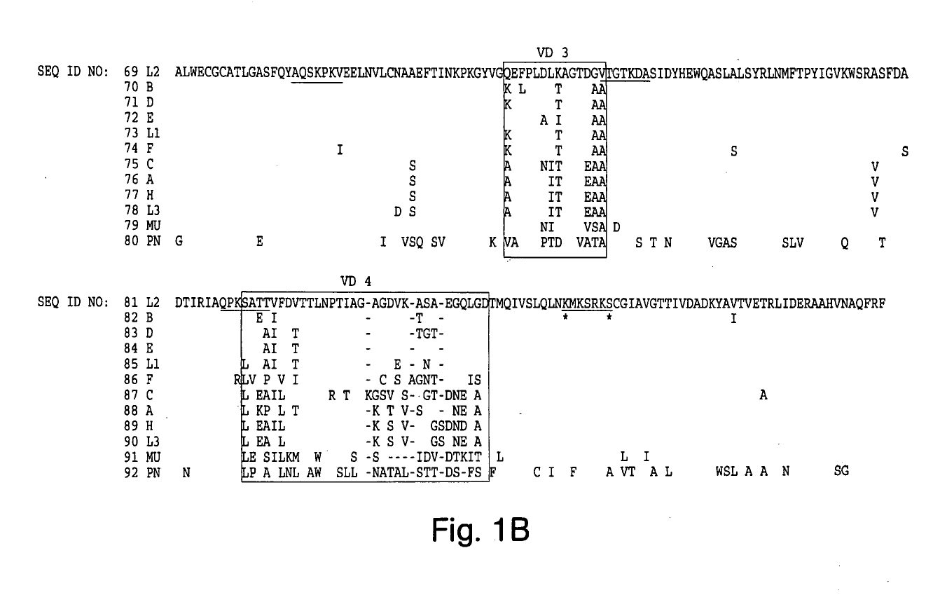

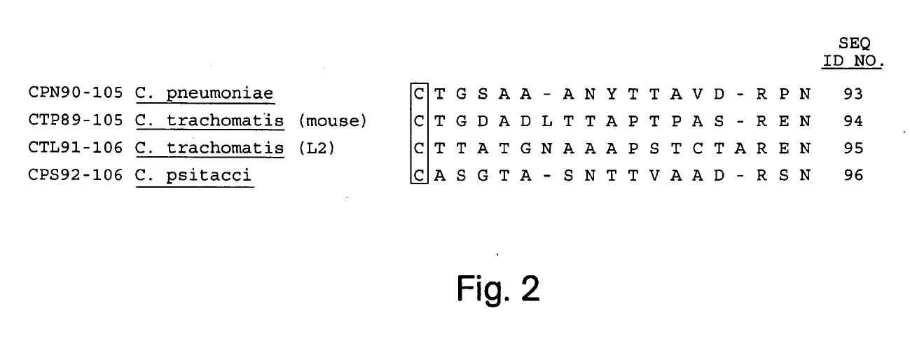

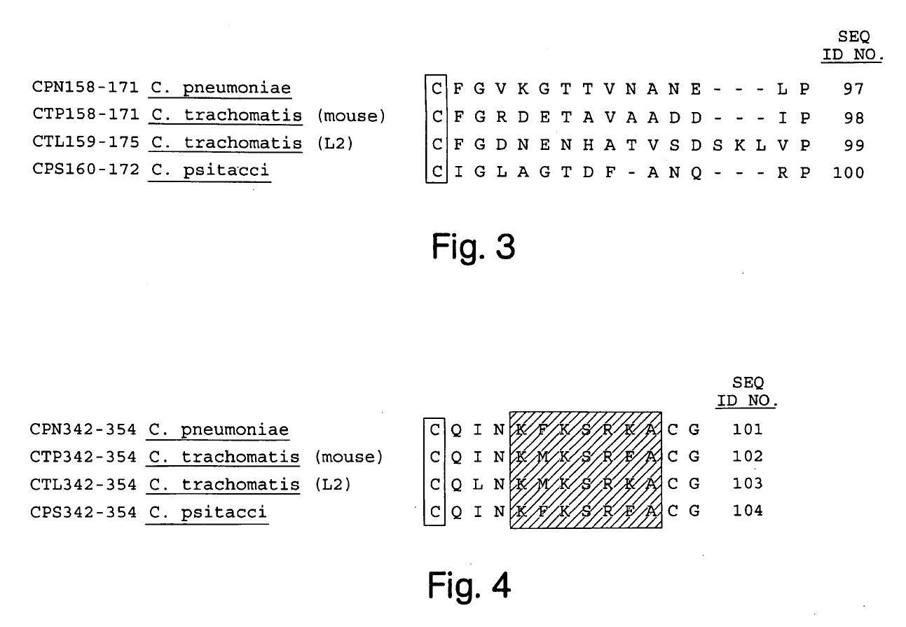

In order to provide ELISA assays that are species- and potentially strain-specific for the various Chlamydia, two regions in the MOMP have been identified which show minimal amino acid sequence homologies and which are predicted to be excellent antigenic domains by virtue of hydrophilicity and peptide mobility on the solvent-accessible surface of MOMP. FIG. 1 illustrates the constant and variable domain (VD) of the various chlamydial species. The identified species-specific antigenic domains are located in VD1 and VD2. FIG. 2 illustrates the peptide amino acid sequences employed for the construction of peptide based ELISAs with species specificity for VD1. FIG. 3 illustrates the peptides for VD2 which are used similarly to the VD1 sequences. In addition, a highly antigenic domain (FIG. 4) common to all Chlamydia has been identified and developed as genus-specific ELISA for the Chlamydia. Immunization of rabbits has verified the antigenicity of each peptide to each peptide (Table 1). Monoclonal antibodies have further verified the specificities and antigenicity of each peptide (Table 1) as predicted by computer analysis of the nucleotide-generated amino acid sequence of each species-specific MOMP.

TABLE 1
Antigenic Responses to Peptides From Four species
Of Chlamydiae Identified By Hydrophilicity And
Peptide Movement As Highly Antigenic

| | |
|---|---|
| Chlamydiae | Titer^{a} |

| Species | Peptide^{b} | Pre | Post |
|---|---|---|---|
| C. pneumoniae | 90-105 | 100 | >3200 |
| C. trachomatis L2 | 91-106 | 800 | >3200 |
| C. psittaci | 92-106 | 400 | >3200 |
| C. trachomatis (mouse) | 89-105 | 0 | >3200 |
| C. pneumoniae | 158-171 | 25 | >3200 |
| C. trachomatis L2 | 159-175 | 200 | >3200 |
| C. psittaci | 160-172 | 100 | >3200 |
| C. trachomatis (mouse) | 158-171 | 800 | >3200 |
| C. pneumoniae | 342-354 | 200 | >3200 |
| C. trachomatis L2 | 342-354 | 100 | >3200 |
| C. psittaci | ND^{c} | | |
| C. trachomatis (mouse) | ND^{c} | | |

^{a}Reciprocal titer
^{b}Immunogenic peptide and ELISA antigen of specific amino acid sequence against the indicated pre-immunization and post-immunization rabbit serum
^{c}ND, not done

Table 2 illustrates reciprocal titers of a polyclonal and monoclonal antibody against C. trachomatis cross-reactive against a C. pneumoniae peptide encompassing amino acids 342-354 and a recombinant full length MOMP from C. pneumoniae. Note that the monoclonal antibody raised against C. trachomatis has as its epitope genus-specific reactivity against peptide 342-354 of C. pneumoniae.

TABLE 2

Titer^{a}

| Antigen | Polyclonal^{b} Ab | Monoclonal Ab^{c} |
|---|---|---|
| CPN Momp^{d} | 400 | 0 |
| CPN 90-105^{e} | 50 | 0 |
| CPN 158-171^{f} | 50 | 0 |
| CPN 342-354^{g} | >3200 | 1600 |

^{a}Reciprocal titer
^{b}Polyclonal goat Ab from Chemicon International, Inc. (Temecula, CA) against MOMP of C. trachomatis
^{c}Monoclonal Ab from ICN Immunologicals (Costa Mesa, CA) against MOMP of C. trachomatis
^{d}C. pneumoniae recombinant MOMP
^{e}Amino acid peptide 90-105 of C. pneumoniae
^{f}Amino acid peptide 158-171 of C. pneumoniae
^{g}Amino acid peptide 342-354 of C. pneumoniae

2) Antigenicity of the 76 kD Protein of C. pneumoniae

C. pneumoniae expresses a gene encoding a unique 76 kD protein (Perez-Melgosa et al., Infect. Immun. 62:880-886 (1994)). Hydrophilicity/peptide flexibility analysis predicts the sequence of amino acids 302-315 (KPKESKTDSVERWS; SEQ ID NO: 1) to be highly antigenic; the peptide has been extended towards the carboxyl terminus to include aromatic and additional hydrophilic amino acid residues. The predicted sequence has been further modified to include an adjacent relatively hydrophilic region containing an aromatic amino acid (tryptophan). Other potential antigenic peptides based on either hydrophilicity or peptide flexibility and extended to include amino acids found in hydrophilic or flexible segments, as well as inclusion of aromatic amino acids immediately adjacent to the predicted antigens, are illustrated in Table 3.

TABLE 3
a) Peptide Movement Predictions

| | | |
|---|---|---|
| SSNSSSSTSRS | (SEQ ID NO: 2) | AA 335-345 |
| GSKQQGSS | (SEQ ID NO: 3) | AA 599-606 |
| GKAGQQQG | (SEQ ID NO: 4) | AA 683-690 |
| PSETSTTEK | (SEQ ID NO: 5) | AA 35-43 |
| KPADGSDV | (SEQ ID NO: 6) | AA 583-590 |
| NGQKKPLYLYG | (SEQ ID NO: 7) | AA 70-80 |
| SDVPNPGTTVGGSKQQGSS | (SEQ ID NO: 8) | AA 588-606 |
| HMFNTENPDSQAAQQ | (SEQ ID NO: 9) | AA 636-650 |

b) Hydrophilic Prediction

| | | |
|---|---|---|
| DDAENETAS | (SEQ ID NO: 10) | AA 617-625 |

3) Antigenicity of the Chlamydial Heat Shock Proteins

C. pneumoniae expresses three known genes with significant homology to the human heat shock proteins of 70, 60 and 10 kD. Antigenicity of homologous regions may result in molecular mimicry and autoimmunity. Indeed, it is postulated that the tubal scarring secondary to infection from C. trachomatis is due to cross-reactive cell mediated immunity against one or more heat shock proteins.

a) C. pneumonia DNAK/Heatshock Protein 70:

Hydrophilicity/peptide flexibility analysis predicts a highly antigenic sequence in the C-terminal region of the expressed protein. This antigenic domain and its homologous human protein are illustrated in Table 4; vertical lines indicate residue homology while “+” signs indicate retention of a positive charge at the site. Amino acid residues 522-529 are either homologous to the human protein or possess preservation of charge (i.e., AA 525-529). Antibodies against this epitope would be expected to possess cross-reactivity with the human 70 kD heat shock protein. Peptides incorporating the C-terminal end of this common region with the non-homologous sequence would be expected to identify Chlamydial-specific antibodies. Two embodiments of this invention include the full length peptide (AA 521-536) and the Chlamydial-specific epitopic sequence identified as AA 527-536 or truncated for the identification of Chlamydia-specific antibodies. Table 5 illustrates other potential antigenic sequences for the DNAK protein expressed by C. pneumoniae based on either peptide flexibility or hydrophilicity and extended to include amino acids found in adjacent hydrophilic or flexible segments, as well as inclusion of aromatic amino acids immediately adjacent to the predicted antigens.

TABLE 4

| | | |
|---|---|---|
| C. pneumoniae (AA 521-536) | KEEDKKRREASDAKNE | (SEQ ID NO: 11) |
| | +\|\|\|++++\| \| | |
| Human hsp70 (AA 569-584) | AEEDRRKKERVEAVNM | (SEQ ID NO: 12) |

TABLE 5

| | | |
|---|---|---|
| KKHSFSTKPPSNNGSSEDHIE | (SEQ ID NO: 13) | (AA 628-649) |
| E | | |
| YTVTSGSKGDAVFE | (SEQ ID NO: 14) | (AA 94-107) |
| TSSEGTRTTPS | (SEQ ID NO: 15) | (AA 34-44) |
| SEHKKSSK | (SEQ ID NO: 16) | (AA 2-9) |
| KDVASGKEQKIRIE | (SEQ ID NO: 17) | (AA 487-500) |
| ERNTTIPTQKKQIFST | (SEQ ID NO: 18) | (AA 411-426) |
| YFNDSQRASSTKDAGR | (SEQ ID NO: 19) | (AA 148-162) |
| EEFKKQEGIDLSKDN | (SEQ ID NO: 20) | (AA 240-254) |
| NAKGGPNINTED | (SEQ ID NO: 21) | (AA 615-626) |
| GERPMAKDNKETGRFD | (SEQ ID NO: 22) | (AA 441-456) |

b) C. pneumoniae GROEL/Heatshock Protein (hsp 60) 60:

Two peptides expressed by the GROEL gene of C. pneumoniae have a high correlation of hydrophilicity and segment mobility (Table 6). Residues with similar negative charges are identified by “*” symbols. The sequences are highly conserved between C. pneumoniae heat shock protein (hsp) 60 and the human hsp 60 associated with the mitochondrion. Thus the potential for molecular mimicry is high and is a likely site for the development of humoral autoimmune responses. Other potential antigenic regions based on either peptide flexibility or hydrophilicity and extended to include amino acids found in adjacent hydrophilic or flexible peptide segments, as well as inclusion of aromatic amino acids immediately adjacent to the predicted areas, are illustrated in Table 7.

TABLE 6

| | | |
|---|---|---|
| C. pneumoniae hsp 60 (AA 385-398) | TEIEMKEKKDRVDD | (SEQ ID NO: 23) |
| | + \| \|\|\|\|\|\| \| | |
| Human hsp 60 (AA 410-423) | SDVEVNEKKDRVTD | (SEQ ID NO: 24) |
| C. pneumoniae hsp 60 (AA 354-364) | EDSTSDYDKEK | (SEQ ID NO: 25) |
| | + \|\|+\|+\|\|\| | |
| Human hsp 60 (AA 410-420) | DVTTSEYEKEK | (SEQ ID NO: 26) |

TABLE 7

| | | |
|---|---|---|
| DDKSSSA | (SEQ ID NO: 27) | (AA 528-534) |
| KKQIEDSTSDYVSEE | (SEQ ID NO: 28) | (AA 350-364) |
| SSYFSTNPETQE | (SEQ ID NO: 29) | (AA 201-212) |
| EKVGKNGSITVEEADK | (SEQ ID NO: 30) | (AA 167-182) |
| SKTADKAGDGTTTAT | (SEQ ID NO: 31) | (AA 79-93) |

c) C. pneumoniae GROES/Heat Shock Protein 10 (hsp 10):

Three peptides are highly correlated with respect to hydrophilicity/peptide movement analysis. Comparison to mouse chaperonin 10 indicates little homology of these bacterial antigenic domains with C. pneumoniae hsp 10 (Table 8).

TABLE 8

| | | |
|---|---|---|
| C. pneumoniae (AA 20-29) | KREEEEATAR | (SEQ ID NO: 32) |
| | \| \| + | |
| mouse chaperonin 10 (AA 19-28) | ERSAAETVTK | (SEQ ID NO: 33) |
| C. pneumoniae (AA 36-46) | DTAKKKQDRAE | (SEQ ID NO: 34) |
| | + \| \| | |
| mouse chaperonin 10 (AA 35-45) | EKSQGKVLQAT | (SEQ ID NO: 35) |
| C. pneumoniae (AA 51-60) | GTGKRTDDGT | (SEQ ID NO: 36) |
| | \| \| + \| | |
| mouse chaperonin 10 (AA 50-59) | GSGGKGKSGE | (SEQ ID NO: 37) |

4) Antigenicity of the Cysteine-Rich Proteins of C. pneumoniae

a) 60 KD/OMP B:

The second most abundant protein of the external matrix is a 60 kD protein containing 34 cysteines (6.1%). Table 9 illustrates the single peptide domain with overlapping hydrophilicity and peptide flexibility profiles. The sequence has been extended towards the C-terminus to include additional hydrophilic amino acids and two aromatic residues.

Table 10 illustrates several additional peptides with potential antigenic profiles based on either peptide flexibility or hydrophilicity and extended to include amino acids found in adjacent hydrophilic or flexible peptide segments as well as inclusion of aromatic acids immediately adjacent to the predicted areas.

TABLE 9

| | | |
|---|---|---|
| RNKQPVEQKSRGAFCDKEFYPC | (SEQ ID NO: 38) | (AA 60-84) |
| EE | | |

TABLE 10

| | | |
|---|---|---|
| DMRPGDKKVFTVEFCPQRR | (SEQ ID NO: 39) | (AA 278-296) |
| SSDPETTPTSDGKVWKIIDR | (SEQ ID NO: 40) | (AA 157-176) |
| TSESNCGTCTSCAETTTHWK | (SEQ ID NO: 41) | (AA 418-437) |
| KLGSKESVEFS | (SEQ ID NO: 42) | (AA 511-521) |
| TVYRICVTNRGSAEDT | (SEQ ID NO: 43) | (AA 459-474) |
| EYSISVSNIPGD | (SEQ ID NO: 44) | (AA 343-353) |

b) 9 kD Protein:

This small protein contains 14 cysteines (15.5%). Table 11 illustrates the predicted antigenic sites. Peptide 1 represents the single peptide for the 9 kD cysteine-rich protein identified by common hydrophilic/peptide flexibility profiles. Peptide 2 recognized initially by its peptide flexibility and extended towards the amino terminal to include several hydrophilic residues.

TABLE 11

| | | |
|---|---|---|
| Peptide 1: RKKERS | (SEQ ID NO: 105) | (AA 44-49) |
| Peptide 2: STECNSQSPQ | (SEQ ID NO: 106) (AA 68-77) | |

5) Antigenicity of the Ebola Virus GP Protein:

The GP protein associates into trimers on the surface of the virus and functions as an attachment protein. Two peptides are predicted to be excellent antigens on the basis of overlapping hydrophilic/peptide flexibility profiles (Table 12). Additional potential antigenic sites initially based on either peptide flexibility or hydrophilicity and extended to include amino acids found in adjacent hydrophilic or flexible peptide segments as well as inclusion of aromatic amino acids immediately adjacent to the predicted domains are illustrated in Table 13.

TABLE 12

| | | |
|---|---|---|
| NPNLHYWTTQDEG | (SEQ ID NO: 107) | (AA 512-524) |
| SGQSPARTSSDPGTNTTTEDH | (SEQ ID NO: 108) | (AA 320-340) |
| K | | |

TABLE 13

| | | |
|---|---|---|
| TGGRRTRRE | (SEQ ID NO: 109) | (AA 494-502) |
| RDRFKRTSFF | (SEQ ID NO: 110) | (AA 11-21) |
| EQHHRRTDNDST | (SEQ ID NO: 111) | (AA 405-416) |
| ENTNTSKSTDF | (SEQ ID NO: 112) | (AA 433-443) |
| YTSGKRSNTTGK | (SEQ ID NO: 113) | (AA 261-272) |
| TTTSPQNHSET | (SEQ ID NO: 114) | (AA 448-458) |
| PDQGDNDNWWT | (SEQ ID NO: 115) | (AA 636-646) |
| TISTSPQSLTTK | (SEQ ID NO: 116) | (AA 370-381) |
| TEDPSSGYYSTTIRYQ | (SEQ ID NO: 117) | (AA 206-221) |
| THHQDTGEESASSGK | (SEQ ID NO: 118) | (AA 464-478) |

6) Detection Assay Methods

a. Immunoglobulin (Ig) Assay

C. pneumoniae EBs were grown in primary human umbilical vein endothelial cells (HuEVEC; early passage), HeLa 199, or a suitable alternative in the presence of 1 μg/ml cycloheximide at 35° C. under 5% CO₂. Permissive cells were lysed at 3 days, thereby liberating EBs. The latter were harvested from infection flasks, sonicated, and cellular debris were removed after sonication by a low speed centrifugation (.about.600×g) for 5 minutes. EBs were pelleted by high speed centrifugation (30,000×g) for 30 minutes at 4° C. The EB pellet was washed with PBS×1 and was reconstituted in 2 ml PBS per four 25-cm² culture flask and sonicated at maximum power for 20 seconds and a 0.5 cycle time using a Braun-Sonic U sonicator. EB protein concentration was determined by the Bradford method and the sonicated infectious EB suspension was rendered non-infectious by the addition of 37% formaldehyde to a final 10% formaldehyde concentration with constant agitation during addition. Formalin-treated EBs were added to 96-well plates at 50 μl per well containing 50 ng EB (total of 5 μg/plate) and air dried. The plate was washed with PBS-0.15% Tween20×3 and was then blocked with PBS-1% BSA-0.15% Tween20 at 300 μl per well for 1 hour at room temperature and then washed×3 with PBS-0.15% Tween20. Serum was serially diluted in PBS in duplicate in a separate plate and 50 μl of each well transferred to corresponding wells of a MOMP ligand plate and the following sequence was followed: incubate at 37° C. for 1 hour using a parafilm cover; wash with PBS-1% FCS-0.05% NaN3×5; incubate each well with a predetermined dilution of biotin-conjugated, antihuman monoclonal IgG or monoclonal IgM; incubate at 37° C. for 1 hour with cover; wash (×3) with PBS, 1% FCS, 0.05% NaN3; follow with 50 μl strepavidin-alkaline phosphatase conjugate (1:200 in PBS-1% BSA-0.15% Tween20) for 1 hour at 37° C. with cover; and wash×3 with PBS, 1% CS, 0.05% NaN3. Color was developed with p-nitrophenyl phosphate in glycine buffer at pH 9.6. The color yield was measured on a Flow microtiter calorimeter using a 405 nm filter. End point titer was the highest dilution of serum or secretion yielding a color yield>3 SD over background (n=8).

b. Western Blot

Western blots were prepared by SDS-PAGE of C. pneumoniae EBs (non-formalin fixed) harvested from infected HuEVEC or HeLa cell lysates, electrophoresed under standard SDS-PAGE conditions, and transferred to nitrocellulose achieved with an active diffusion transfer. Albumin-blocked strips were prepared from nitrocellulose sheets and incubated 1 hour with 1.2 ml of a 1:40 dilution of test serum. Detection was achieved with an alkaline phosphatase-conjugated, mouse anti-human antibody, and developed with 5-bromo-4-chloro-3′-indolyphosphate p-toluidine/nitro-blue tetrazolium chloride (BCIP/NBT, Pierce Chemical Company). Polyclonal animal anti-human antibodies can alternatively be used.

c. Antigen Capture Assay for Chlamydial MOMP

The peptides described in FIGS. 2-4 were conjugated via disulfide bonding to keyhole limpet hemocyanin (KLH) by standard methods (Bernatowicz et al., Anal. Biochem. 155(1):95-102 (1986)). The peptide conjugates in alum were used to generate polyclonal and/or monoclonal antibodies to the species-specific domains of MOMP which is used as a capture antibody in 96 well microtiter plates. Final configuration can follow a number of alternative routes to yield quantitation of MOMP in body fluids. The favored configuration utilizes biotin labeled recombinant MOMP in a competition assay with strepavidin/alkaline phosphatase generated color development based on the quantity of biotinylated recombinant MOMP displaced by unlabeled MOMP in body fluids.

Equivalents

Those skilled in the art will recognize, or be able to ascertain, using no more than routine experimentation many equivalents to the specific embodiments of the invention described herein. Such equivalents are intended to be encompassed by the following claims.

What we claim is:

1. A substantially pure antibody that specifically binds to a polypeptide consisting essentially of SEQ ID NO: 93 or SEQ ID NO: 101.

2. The antibody of claim 1, wherein said antibody is a monoclonal antibody.

3. A method of preparing the antibody that specifically binds to a polypeptide consisting essentially of SEQ ID NO: 93 or SEQ ID NO: 101, comprising said method comprising the steps of:
(a) immunizing an animal with a polypeptide consisting essentially of SEQ ID NO: 93 or SEQ ID NO: 101; and
(b) recovering an antibody that specifically binds to a polypeptide consisting essentially of SEQ ID NO: 93 or SEQ ID NO: 101 from the animal.

4. A method of identifying the presence of Chlamydia pneumoniae in a patient, said method comprising the steps of:
(a) providing a biological sample from said patient;
(b) contacting said sample with an antibody that specifically binds to a polypeptide consisting essentially of SEQ ID NO: 93 or SEQ ID NO: 101 under conditions that allow binding between said antibody and Chlamydia pneumoniae;
(c) removing unbound antibody; and
(d) detecting the presence of antibody bound to said sample, wherein the presence of antibody bound to said sample indicates the presence of Chlamydia pneumoniae in said patient.
